(19) United States
(12) Patent Application Publication
(10) Pub. No.: US 2011/0152643 A1
(43) Pub. Date: Jun. 23, 2011

(54) "Band-aid"-type potassium ion (K+) biosensor

(76) Inventors: Ruipeng Xue, Columbus, OH (US); Pelagia Gouma, Port Jefferson, NY (US)

(21) Appl. No.: 12/903,157

(22) Filed: Oct. 12, 2010

Related U.S. Application Data
(60) Provisional application No. 61/251,269, filed on Oct. 13, 2009.

(51) Int. Cl. A61B 5/00

(52) U.S. Cl. 600/309

(57) ABSTRACT

Potassium ion (K+) is important in regulating normal cell function in the human body, specifically the heartbeat and the muscle function. It is important to be able to monitor potassium ion concentrations in human fluids. This invention describes a novel concept for a potassium ion biosensor that accurately, rapidly, and efficiently monitors the presence and records the concentration of potassium ions with high specificity, not only in serum and urine, but also in the sweat or even eye fluid. This specific biosensor design utilizes a nanomanufacturing technique, i.e. electrospinning, to produce advanced nano-bio-composites that specifically trace even minute quantities of potassium ions through the use of selective bio-receptors (ionophores) attached to high surface area nanofibers. Electroactive polymers are then employed as transducers to produce an electronic (rather than ionic) output that changes instantly with the change in K+ concentration. Such biosensors may be manufactured in a skin patch configuration.

### PRIORITY

This utility application claims the priority of provisional application 61/251,269 filed with the U.S. Patent and Trademark Office on Oct. 13, 2009, the contents of which is incorporated herein by reference.

### BACKGROUND OF THE INVENTION

Potassium ion (K+) is important in regulating normal cell function in the human body, specifically the heartbeat and the muscle function. Thus, it is important to be able to monitor potassium ion concentrations in human fluids. This invention describes a novel concept for a potassium ion biosensor that accurately, rapidly, and efficiently monitors the presence and records the concentration of potassium ions with high specificity, not only in serum and urine, but also in the sweat or even eye fluid. This specific biosensor design utilizes a nanomanufacturing technique, i.e. electrospinning, to produce advanced nano-bio-composites that specifically trace even minute quantities of potassium ions through the use of selective bio-receptors (ionophores) attached to high surface area nanofibers. Electroactive polymers are then employed as transducers to produce an electronic (rather than ionic) output that changes instantly with the change in K+ concentration. Such biosensors may be manufactured in a skin patch configuration.

Electrospinning is a fiber forming process capable of producing non-woven mats of nanoscale dimensios, thus offering large surface area to volume ratio, and controlled pore size distribution. During the process a polymer solution or composite mixture (soluble drugs, bacterial agents, and metal oxide sol-gel solutions may be added to the polymer) is injected from a small nozzle under the influence of a strong electric field. The build up of electrostatic charges on the surface of a liquid droplet induces the formation of a jet. Before the jet reaches the collecting screen, the solvent evaporates or solidifies. The resulting fibers are collected in the form of a non-woven mat. The setup for electrospinning consists of four major components: a high-voltage power supply (Gamma High Voltage), a programmable syringe pump (Kd Scientific), a syringe, needle (Popper & Sons) and a grounded collector screen.

In our experiments, Polyvinylpyrrolidone (PVP) powder, with an average molecular weight of 1,300,000 g/mol, and ethanol were mixed at room temperature. The solutions were magnetically stirred for about 1 hour and then introduced into the syringe. The disposable 5 ml syringe and 22 G needle were used for all experiments. The solution concentrations were varied to investigate their effects on the morphology of the electrospun mats. The electrospun fibers were collected on an aluminum foil to form fiber membranes.

### BRIEF SUMMARY OF THE INVENTION

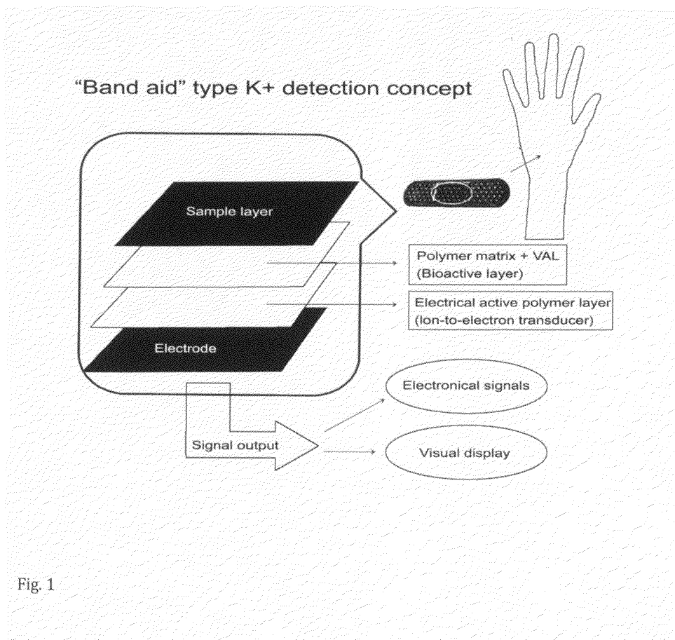

We have invented the concept of a “band-aid” type sweat test for measuring potassium ion levels fast, accurately, and potentially wirelessly. Measuring K⁺ concentration in sweat is expected to overcome the problems/artifacts often encountered during the blood drawing process altering the level of (extracellular) potassium concentration measured in serum (see pseudohyperkalemia [1]). Based on the medical literature, there are known levels of normal potassium concentration in the sweat of humans (4.7-9.7 mM/L for the male population; 7.6-15.6 mM/L for the female population [2]). Using this knowledge, a convenient, non-invasive, maintenance-free, sensitive and inexpensive potassium ion monitor is described (see FIG. 1).

FIG. 1 illustrates the layered configuration of the proposed skin patch for sweat testing. Similarly to the drug sweat test [3], the patient will wear the patch for long periods of time so there is no need to activate the release of sweat, as is the case with the sweat test for cystic fibrosis which requires a large amount of sweat sample [4]. The first layer in contact with the skin consists of a non-woven carrier membrane on which the ionophore is positioned; this ionophore selectively binds with potassium ions and transfers them to the second membrane underneath, the ion-to-electron transducer based on electroactive polymer composites. Finally, a layer of electrodes are incorporated on the external surface of the flexible monitor (patch) to enable (wireless) transmission of the signal for (remote) data analysis, as well as potentially visual display of the electrolyte's concentration.

### DETAILED DESCRIPTION OF THE SEVERAL VIEWS OF THE DRAWING

FIG. 1 depicts the “band-aid”-type potassium ion monitor concept; note the layered configuration of the proposed skin patch for sweat testing.

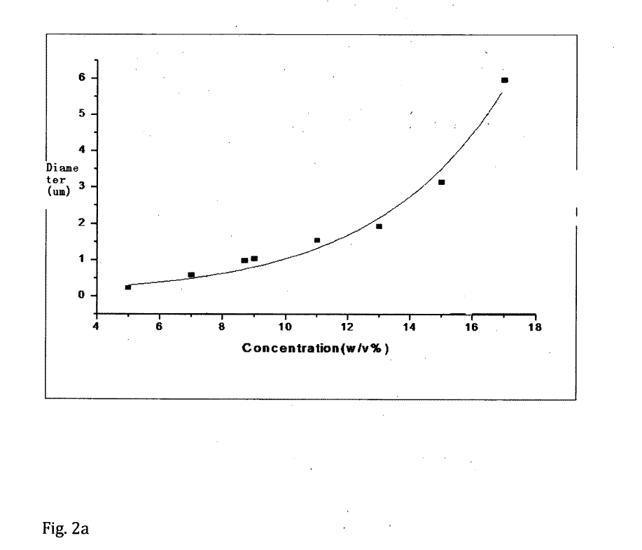

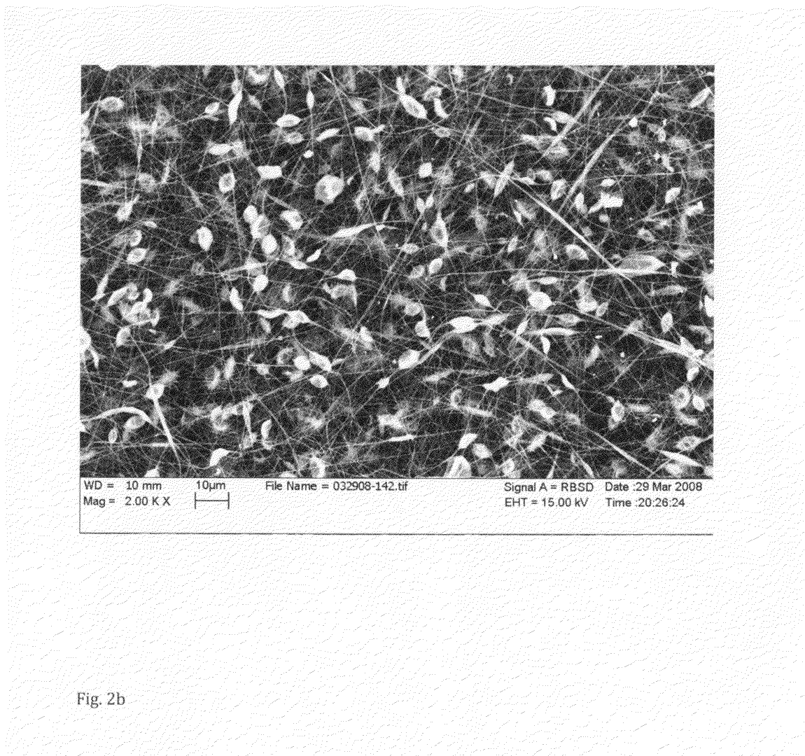

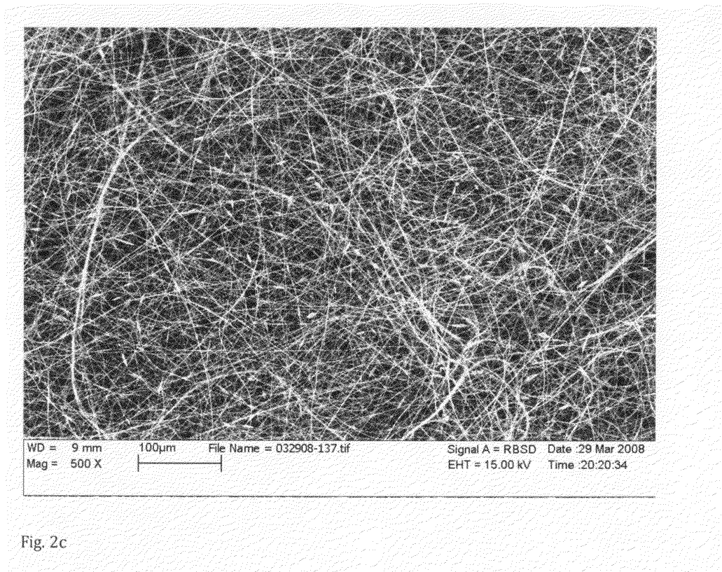

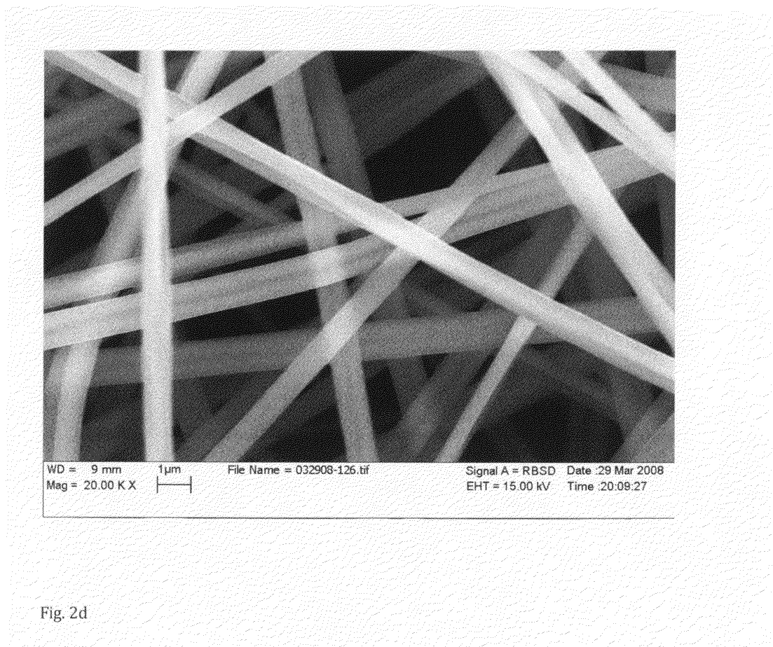

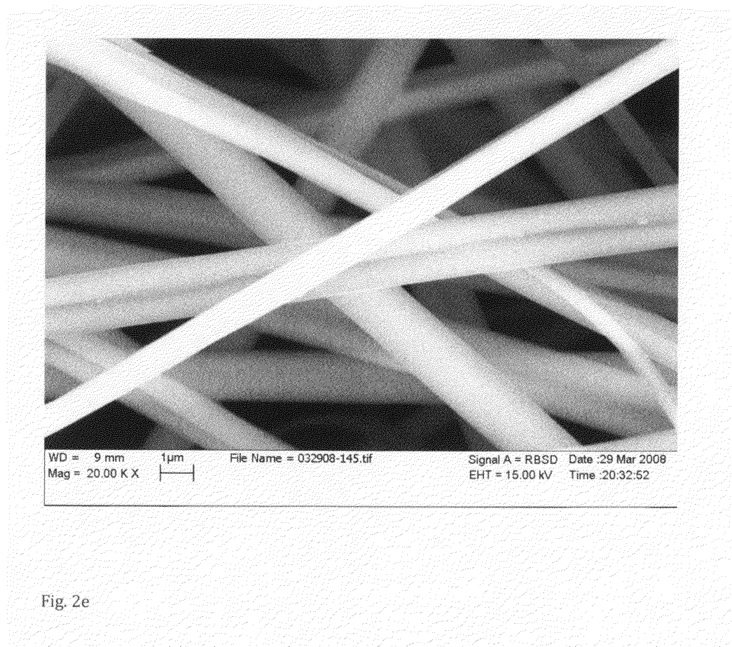

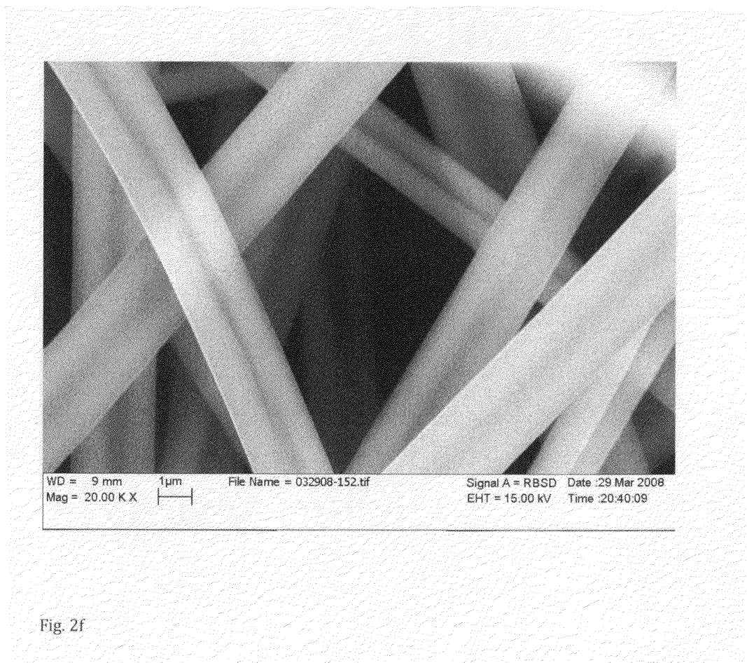

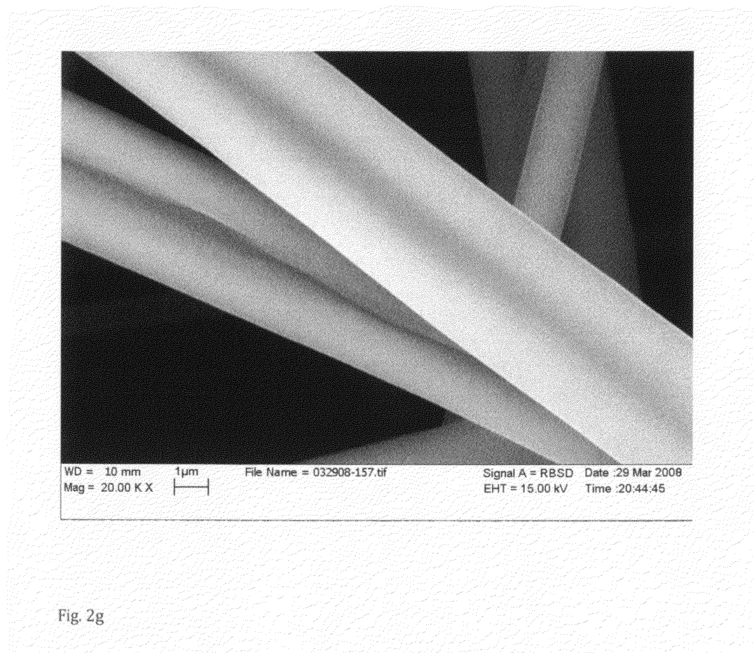

FIG. 2a illustrates the process-map obtained from experiments for the bio-nanocomposite mats (non-woven carrier membrane)

FIG. 2b illustrates the morphology of the nonwoven carrier membrane at 5% concentration of the precursor solution

FIG. 2c illustrates the morphology of the nonwoven carrier membrane at 7% concentration of the precursor solution

FIG. 2d illustrates the morphology of the nonwoven carrier membrane at 8.7% concentration of the precursor solution

FIG. 2e illustrates the morphology of the nonwoven carrier membrane at 13% concentration of the precursor solution

FIG. 2f illustrates the morphology of the nonwoven carrier membrane at 15% concentration of the precursor solution

FIG. 2g illustrates the morphology of the nonwoven carrier membrane at 17% concentration of the precursor solution

### DETAILED DESCRIPTION OF THE INVENTION

Below we introduce each of the components of the biosensor separately:

I. BIO-ACTIVE NANOFIBROUS MATS

We have produced process maps for various precursor (polymer concentration) and process conditions (flow rate, voltage and working distance), for the given potassium ion sensing system (the precursor solution consisted of different ratios of polyvinylpyrrolidone (PVP) dissolved in ethanol with/without valinomycin—Potassium ionophore I-solutions) as shown in FIG. 2a. Valinomycin is a member of the group of natural neutral ionophores, i.e. it doesn't have a residual charge and is highly selective for potassium. It is a dodecadepsipeptide, made of twelve alternating amino acids and esters to form a macrocyclic molecule. The diameter of valinomycin is about 2 nm.

It is important to note that most PVP electrospun fibers obtained in this work have folded ribbon-like cross-sections. The formation of this kind of fibers is closely related to the solvent evaporation. Generally, the rapid evaporation of solvent “in-flight” makes the fiber surface region rich with polymer and the so-called “skin” forms. As time progresses, the skin layer grows thicker. If the evaporation rate is slow enough, the fiber would be uniform and therefore the cross-section would be circular. If the solvent evaporation rate is fast, collapsed hollow tubes or ribbon like fibers form. These fibers are fundamentally different from the common cylinder-shaped electrospun fibers. The spacing may be tailored to act as anchoring site to accommodate the valinomycin molecules. (The size of the spacing changes with the size of the fiber and it is about one fifth of the fiber width.) FIGS. 2b-2g below illustrates the various fiber mat morphologies obtained (using scanning electron microscopy) by varying the concentration of the precursor solution.

II. ION-TO-ELECTRON TRANSDUCER

Conducting polymers were first introduced as promising ion-to-electron transducers in potassium ion selective electrodes (ISEs) [5]. However, such electrodes still have limited specificity to K+, thus such biosensors are not attractive. The ion-to-electron transduction process in electrospun polyaniline mats has previously been studied by A.Bishop-Haynes [Ph.D. thesis, SUNY Stony Brook, 2008]. Thus, by combining the transducing ability of electroactive polymers, such as polyaniline (PANI) [5], with the selective potassium ion detection offered by the sensing mat described above, chemical information (potassium ion concentration) is converted into electrical signal in the solid state. Thus our biosensor concept becomes a resistive solid-state chemodetector, giving a robust, accurate, and fast response to the changes in potassium ion concentration. It is also easily interfacing with electronic circuits and wireless platforms.

### REFERENCES

1. Walker, H. K. et al, Clinical Methods: The History, Physical, and Laboratory Examinations, editors Stoneham (MA): Butterworth Publishers, (1990)

2. http://www.liv.ac.uk/˜agmclen/Medpracs/practical_2/practical_2.pdf

3. http://www.drugpolicy.org/law/drugtesting/sweatpatch_/

4. Vicky, A. et al. “ Diagnostic and Therapeutic Methods: Sweat analysis proficiency testing for cystic fibrosis.” Pediatric Pulmonology Vol 30, Issue 6, (2000) pp476-480

5. Lindfors, T .et al. “Stability of the inner polyaniline solid contact layer in all-solid-state K+ selective electrodes based on plasticized poly(vinyl chloride)” Analytical Chemistry Vol 76, (2004), pp4387-4394

What is claimed is:

1. This invention claims a device for the monitoring of potassium ion concentration in sweat and/or eye fluid, and/or urine, and/or serum.

2. The device of claim 1 whereas the device is a skin patch.

3. The device of claim 1 whereas the potassium ion sensor is a bio-active nanofibrous mat.

4. The device of claim 1 whereas the biosensor is produced by the electrospinning processing of polyvinylpyrrolidone powder dissolved in ethanol, and (ionophore) valinomycin solutions together.

5. The device of claim 1 whereas an ion-to-electron transducer is also used.

6. The device of claim 1 whereas the ion-to-electron transducer may be electro-active polymer such as polyaniline (PANI).

7. The device of claim 1 whereas PANI may be processed by electrospinning.

8. The device of claim 1 whereas this device can be a resistive chemodetector.

9. The device of claim 1 whereas the sensor output is transmitted wirelessly.

10. The device of claim 1 whereas this device is a medical diagnostics tool.
